(19) United States
(12) Patent Application Publication
(10) Pub. No.: US 2022/0000095 A1
(43) Pub. Date: Jan. 6, 2022

(54) ERYTHROCYTE MEMBRANE COATING

(71) Applicant: Florida Atlantic University Board of Trustees, Boca Raton, FL (US)

(72) Inventors: Peng YI, Boca Raton, FL (US); Tanaz ISLAM, Kelowna (CA)

(21) Appl. No.: 17/366,187

(22) Filed: Jul. 2, 2021

Related U.S. Application Data
(60) Provisional application No. 63/047,314, filed on Jul. 2, 2020.

(51) Int. Cl. A01N 1/02

(52) U.S. Cl. CPC A01N 1/0226

(57) ABSTRACT

A novel erythrocyte (red blood cell) membrane coating derived from human red blood cells and the methods of preparation and use thereof are disclosed. The erythrocyte membrane coating may be developed on a piezoelectric sensor coated with poly-L-lysine.

### CROSS-REFERENCE TO RELATED APPLICATIONS

This non-provisional application claims the benefit under 35 U.S.C. § 119(e) of U.S. Provisional Application Ser. No. 63/047,314 filed on Jul. 2, 2020 entitled A NOVEL ERYTHROCYTE MEMBRANE COATING and whose entire disclosure is incorporated by reference herein.

### FIELD OF INVENTION

This invention relates to an erythrocyte (red blood cell) membrane coating derived from human red blood cells for use in various applications, such as a toxicity screening for nanoparticles, nanodrug delivery, or as a biosensor. It also relates to the method of forming an erythrocyte membrane coating on a surface of interest.

### BACKGROUND OF THE INVENTION

The increased use of nanoparticles (NPs) in food additives, nanodrugs, and cosmetics can cause them to enter human circulatory system and attach to human cells, particularly red blood cells (RBCs). Such attachment may lead to cytotoxic effects on RBCs. Hence, it is critical to investigate the attachment probability of NPs to RBCs.

In recent years, significant advancements have been achieved towards the applications of NPs. NPs have been increasingly used in the pharmaceutical and biotechnology industries for diagnosis, imaging, and targeted drug delivery. For example, orally administered Gastromark™ (silicone-coated superparamagnetic iron oxide NPs), has been used as a contrast agent in gastrointestinal magnetic resonance imaging (MRI). Ferumoxytol, also known as Feraheme™, (carbohydrate-coated Fe₃O₄ NPs) has been approved for intravenous administration for the treatment of iron deficiency anemia for adult patients with chronic kidney disease. Liposomal nanodrugs like Doxil® and Onivyde® are used as targeted drug delivery for cancer patients. Administration of such drugs into the human body leads to direct contact of red blood cells with these NPs.

Moreover, NPs have been extensively incorporated in various food products (e.g., food additives) and consumer products such as food packaging and cosmetics, likely resulting in the entry of NPs into human digestive systems via different routes. For instance, titanium dioxide (TiO₂) NPs are commonly used in sunscreens, dental implants, and food additives for enhancing food color.

Following entry into the gastrointestinal system, NPs can penetrate through epithelial and endothelial barriers into the bloodstream and lymph stream. Additionally, NPs in diesel soot and carbon black NPs that are released into the air from combustion engines and power plants can enter human respiratory systems, translocate from the lung to the circulation system in the human body, and easily be distributed to lymph nodes, liver, heart, kidney, and brain.

The NPs that enter the digestive, respiratory, and circulatory system would inevitably have contact with and attach to human cells such as epithelial cells, endothelial cells, and various blood cells (i.e., red blood cells, white blood cells, and platelets). Such attachment of nanoparticles to cell membranes has been proposed to be the initial step for NPs to exert cytotoxic effects, since it is a critical step toward the disruption of cell membrane and cellular processes.

AshaRani et al. shows that attachment of silver NPs (AgNPs) to lung fibroblast cells induces cell membrane injury and endocytosis through which AgNPs enter the cells, resulting in the production of reactive oxygen species (ROS). This further results in mitochondria damage, deoxyribonucleic acid (DNA) damage and cell cycle arrest. The attachment of hematite NPs (HemNPs) to human epithelial cell lines is shown to be a critical step for the uptake of HemNPs by the cells, which results in loss of membrane integrity and release of cytokines (i.e., interleukin-6 and interleukin-8) that are known to promote inflammatory responses.

It has also been reported that the contact of HemNPs with myoblast cancer cells form small pores in the membrane through which NPs enter and damage organelles, triggering cell death and apoptosis of cells. In addition, adsorption of polystyrene NPs (PSNPs), commonly used as model plastic NPs, on human intestinal epithelium cells has resulted in cellular internalization of PSNPs and partial colocalization of PSNPs in lysosome, raising concerns on the chronic effects of ingested plastic NPs. In order to estimate the cytotoxic effects of NPs on human cells, it is crucial to first measure the probability of NPs attaching to the membranes of human cells.

In particular, erythrocytes or red blood cells (RBCs), a dominant type of blood cell having the highest chance to encounter the NPs present in the blood stream, is vulnerable to toxicity like deformation, agglutination and membrane damage when they come to contact with NPs. For example, attachment of PSNPs to RBCs was found to increase the osmotic, mechanical and oxidative stress which resulted in sensitization and cell damage of RBCs.

Several methodologies have been proposed to utilize RBCs as drug carriers for the targeted delivery of nanodrugs in the human body, due to the bioavailability, biocompatibility, and longevity of RBCs in the circulation system. Brenner et al., use the surface of RBCs as a hitchhiking tool on which nanocarriers (e.g., nanogel, liposomes, etc.) are adsorbed and transported to the first organ downstream of the intravascular injection. In another study, Doxorubicin loaded poly (lactic-co-glycolic acid) (PLGA) NPs are hitchhiked onto the surface of RBCs for targeted delivery of the chemotherapy for lung metastasis treatment. Thus, from both perspectives of nanoparticle toxicity and nanodrug delivery, it is important to quantitatively study the attachment of NPs to the surface of RBCs (i.e., the membrane of RBCs).

The red blood cell membrane (RBCm), which is derived from whole red blood cells, is used in a variety of applications. For example, an electrochemical enzymatic biosensor has been developed by coating the RBCm on an Au-screen printed electrode for highly selective glucose measurement. An RBCm coating with multi-lamellar stacks of human RBC membranes on hydrophilic and hydrophobic silica chips has also been developed. RBCm was also utilized for blood group determination using immuno-sensor. Specific binding reagents for the blood group antigens were immobilized on the piezoelectric transducer for carrying out an immunological detection method for the determination of blood group.

Some prior research works studied the adsorption (or attachment) of different NPs on the surface of whole RBCs using various observational tools such as scanning electron microscopy (SEM), conventional optical microscopy, transmission electron microscopy (TEM), and confocal laser scanning microscopy (CLSM). For instance, TEM has been used to locate TiO₂ (˜20 nm) aggregates attached on the membrane of and also within RBCs. SEM has been employed to visualize the distribution of 15 nm TiO₂ over the RBC surface. The attachment of polystyrene NPs (200 nm) to RBCs has also been observed using SEM after incubation at the particle/RBC ratio up to 100:1. Nevertheless, quantitative information on neither the mass deposition nor the probability of NPs' attachment on the surface (i.e., cell membranes) of real human cells is still rare due to the lack of appropriate tools.

Recently, piezoelectric sensors, e.g., quartz crystal microbalance (QCM) sensors coated with supported lipid bilayer (SLBs) of synthetic phospholipids such as palmitoyl-2-oleoyl-snglycero-3-phosphocholine (POPC), 1,2-dioleoyl-sn-glycero-3-phosphocholine (DOPC), etc. have been used to conduct quantitative studies on interactions between NPs and model cell membranes. For example, Yi and Chen made DOPC SLBs on poly-L-lysine (PLL) modified silica-coated QCM crystals for studying the deposition kinetics of carboxylated multiwalled carbon nanotubes on model cell membranes. Some recent advancement has been seen in this field by incorporating sterols, charged lipids such as phosphatidylinositol, phosphatidylserine, phosphatidylglycerol, and cell penetrating peptides in SLBs. Moreover, Melby et al. formed a lipid raft system incorporating highly ordered domains of sphingomyelin and cholesterol in a DOPC SLB. However, such SLBs still cannot fully replicate the complex characteristics of real cell membrane of a mammalian cell.

Some early efforts have been made in fixing real cell membranes on the surface of substrates (e.g., electrochemical sensors and silica chips). Himbert et al. developed a highly oriented, multi-lamellar solid supported RBC membranes on silica chips to study the molecular structure of RBC membrane. The RBC vesicles were prepared via hypotonic treatment and sonication and deposited on functionalized (hydrophilic and hydrophobic) silica chips and was annealed at 50° C. and a humidity of 95.8±0.5% in a saturated K₂SO₄ solution for 5 days.

In another study, an RBC membrane layer was used as a diffusive layer to develop an electrochemical sensor for highly selective glucose measurement. An enzyme composite was deposited on Au-screen printed electrode and RBC vesicles were evenly cast on the modified surface of electrode. The electrode was incubated with both enzyme composite and the RBC membranes at 50° C. for 50 min in a dry oven to fabricate the sensor.

However, the aforementioned supported RBCm layers were formed at a high temperature (i.e., 50° C.). Such a high temperature can potentially increase the membrane fluidity, and lead to an irreversible change in membrane elasticity, protein denaturation and enzyme inactivation. Thus, the original biological features of RBCm may have been lost in those layers.

Accordingly, it is desired to provide an RBCm coating that retains the biological features of the red blood cell membrane.

All references cited herein are incorporated herein by reference in their entireties.

### BRIEF SUMMARY OF THE INVENTION

A first aspect of the invention is a supported red blood cell membrane (SRBCm) that has been developed on piezoelectric sensor (i.e., QCM crystals) in aqueous solution at room temperature from human RBCs. It is an unexpected advantage of the present invention that these developed SRBCms retain the biological features of real RBCm.

In certain embodiments, the invention is a coating that may be formed on any analytic substrate, the coating comprising RBCm fragments.

In certain embodiments, the RBCm fragments form a continuous layer on a surface of the analytic substrate.

In certain embodiments, the supported RBCm layer is developed on a silica-coated piezoelectric sensor with negatively charged surface of a quartz crystal microbalance with dissipation monitoring (QCM-D) instrument.

In certain embodiments, the sensor surface is completely coated with a cationic layer of poly-L-lysine (PLL).

In certain embodiments, the coating forms on the surface of the analytic substrate in an aqueous solution.

In certain embodiments, the coating forms on the surface of the analytic substrate without raising the temperature higher than about 37° C.

In certain embodiments, the coating forms on the surface of the analytic substrate without a drying process.

A second aspect of the invention is a method of coating a surface of an analytic substrate with a coating comprising RBCm fragments.

In certain embodiments, the method comprises soaking the surface of the analytic substrate in a suspension of RBCm fragments at room temperature.

In certain embodiments, the method does not comprise a drying process.

### BRIEF DESCRIPTION OF THE DRAWINGS

The invention will be described in conjunction with the following drawings, wherein:

FIG. 1 is a representation of the formation of supported erythrocyte membrane on a silica-coated piezoelectric sensor.

FIG. 2 is a graph depicting frequency and dissipation responses, which were obtained by the QCM-D during the formation process of supported erythrocyte membrane on PLL-modified silica sensor at 1 mM NaCl and 0.2 mM NaHCO₃, pH 7.1.

FIG. 3A is a graph depicting frequency and dissipation responses during deposition of 5 mg/L PSNPs on supported RBCm-PLL-modified surface.

FIG. 3B is a graph depicting frequency and dissipation responses during deposition of 5 mg/L PSNPs on PLL-modified sensor surface in a background solution of 1 mM NaCl and 0.2 mM NaHCO₃. The contrast between no deposition on RBCm and favorable deposition of PSNPs on PLL proved complete coverage of supported RBCm on PLL-modified surface.

FIG. 4A is a 2D AFM image characterization of surface morphology of a supported RBCm on a silica crystal sensor of QCM-D in a solution of 1 mM NaCl and 0.2 mM NaHCO₃.

FIG. 4B is a graph depicting size distribution of surface aggregates of RBCm on supported RBCm (SRBCm) summarized from 2D AFM images of supported RBCm.

FIG. 4C is a 3D AFM image of supported RBCm.

FIG. 5A is a graph depicting Δf₍₃₎ and ΔD₍₃₎ of HemNP deposition on SRBCm when α_{A}<0.0002.

FIG. 5B is a graph depicting Δf₍₃₎ and ΔD₍₃₎ of HemNP deposition on SRBCm when α_{A} was in the range of 0.074-0.173.

FIG. 5C is a graph depicting Δf₍₃₎ and ΔD₍₃₎ of HemNP deposition on bare silica sensor. All deposition experiments of HemNPs were conducted at 1 mM NaCl and pH 5.1. The silica surface was rinsed with 1 mM NaCl for the first phase of the experiment and 8.8 mg/L HemNPs were deposited on silica surface 1 mM NaCl for the second phase of the experiment.

FIG. 5D is a graph depicting deposition attachment efficiencies (α_{D}) on SRBCm of 5 mg/L PSNPs at 1 mM NaCl and 0.2 mM NaHCO₃ and 8.8 mg/L HemNPs at 1 mM NaCl. The error bar represents the standard deviation from mean α_{D}.

FIG. 6 is a graph depicting the entire frequency and dissipation profiles of the QCM-D experiment of PSNPs deposition on SRBCm, the deposition stage of which has been shown in FIG. 3a. The stages are denoted by A to F. A: Stable baselines are obtained by rinsing silica surface with DI water; B: Formation of PLL coating on the silica surface; C: Rinsing PLL layer with 1 mM NaCl and 0.2 mM NaHCO₃; D: Formation of SRBCm E: Rinsing SRBCm with 1 mM NaCl and 0.2 mM NaHCO₃. F: 5 mg/L PSNPs in 1 mM NaCl and 0.2 mM NaHCO₃ was introduced and no deposition take place.

FIG. 7 is a graph depicting complete frequency and dissipation profiles during a QCM-D experiment of HemNP deposition on SRBCm which is a duplicate experiment for the data presented in FIG. 5B. The stages of the QCM-D experiment are denoted by A to G. A: Stable baselines are obtained by rinsing silica surface with DI water; B: Formation of PLL coating on the silica surface; C: Rinsing PLL layer with 1 mM NaCl and 0.2 mM NaHCO₃; D: Formation of SRBCm in 1 mM NaCl and 0.2 mM NaHCO₃ ; E: Rinsing SRBCm with 1 mM NaCl and 0.2 mM NaHCO₃; F: Rinsing SRBCm with 1 mM NaCl; G: 8 mg/L HemNPs in 1 mM NaCl (pH 5.1) was introduced, and continuous deposition of HemNPs on SRBCm occurred. α_{A} of HemNPs was 0.173.

FIG. 8 is a graph depicting complete profiles of frequency and dissipation of a QCM-D experiment of HemNP deposition SRBCm coated sensor. The stages are denoted by A to G. A: Stable baselines are obtained by rinsing silica surface with DI water; B: Formation of PLL coating on the silica surface; C: Rinsing PLL layer with 1 mM NaCl and 0.2 mM NaHCO₃; D: Formation of SRBCm in 1 mM NaCl and 0.2 mM NaHCO₃; E: Rinsing SRBCm with 1 mM NaCl and 0.2 mM NaHCO₃; F: Rinsing SRBCm with 1 mM NaCl; G: 8 mg/L HemNPs in 1 mM NaCl (pH 5.1) was introduced, and deposition of HemNPs on SRBCm occurred. α_{A} of HemNPs was 0.0002.

FIG. 9 is a graph depicting aggregation attachment efficiencies of HemNPs as functions of NaCl concentration at pH 5.1.

### DETAILED DESCRIPTION OF PREFERRED EMBODIMENTS OF THE INVENTION

In certain embodiments, the present invention comprises a red blood cell membrane (RBCm) coating derived from human red blood cells (RBCs). The RBCm coating is mainly composed of flattened fragments of original RBC membranes and partially composed of aggregates of fragments of RBC membrane derived from human red blood cells. The RBC membrane derived from human red blood cells is formed by a single layer of phospholipid bilayers (lipid bilayers) which is made of two layers of phospholipid molecules integrated with membrane proteins, cholesterol, and proteins channels. Each phospholipid molecule has hydrophilic phosphate head, and two hydrophobic lipid tails. They orient them such that the polar heads reside on the outer surface while tails remain in the interior of each layer.

In certain embodiments, the surface of the original RBC membrane may include, for example, enzymes, ligands or biomolecules that bind with a target molecule such as glucose.

The applications of the supported RBCm coatings of the invention are varied. The RBCm coating can play a pivotal role in quantifying the binding of various biomolecules, drugs, or nanoparticles (NPs) on RBC membrane. For instance, the RBCm coating can be used to scrutinize the toxicity of nanodrugs, nanocarriers and nanoparticles on red blood cell. It is crucial to quantitatively determine the attachment probability of those nanoparticles on RBCs upon collisions. The more likely the NPs attach to the RBCm the more likely the NPs can exert toxic effects on RBCs.

In certain embodiments, the supported RBCm may be used for fabricating new biosensors for measuring the binding affinity of biomolecules (e.g., glucose, insulin, antibodies, proteins, RNA, etc.), drugs (e.g., penicillin, aspirin, ibuprofen, nicotine, caffeine, etc.), alcohol, nanoparticles, and ultrafine particulates found in smoke on red blood cells.

In certain embodiments, the supported RBCm is formed on the surface of an analytic surface. For a negatively charged analytic surface, for example, the negatively charged analytic substrate may be first modified by coating with a positively charged substance, such as poly-L-lysine (PLL), to form a positively charged surface of the analytic substrate, before deposition of the RBCm coating. Positively charged PLL is a synthetic polymer which adsorbs electrostatically from the solution onto the negatively charged surface.

In certain embodiments, the supported RBCm is developed on a piezoelectric sensor such as quartz crystal microbalance (QCM) in aqueous solution at room temperature from human RBCs.

In certain embodiments, the suspension is formed by well dispersed RBC membrane, which is prepared from whole blood and characterized thoroughly using cryogenic transmission electron microscopy.

In certain embodiments, the negatively charged surface is modified with PLL to deposit the RBCm coating on it.

In certain embodiments, the RBCm coating is formed without a drying process, such as any process that can result in the exposure of RBCm coating to the air.

The invention will be illustrated in more detail with reference to the following Examples, but it should be understood that the present invention is not deemed to be limited thereto.

### EXAMPLES

### Example 1. Materials.

Preparation and Characterization of Hematite Nanoparticles (HemNPs) and Carboxylated Polystyrene Nanoparticles (PSNPs). The 4.4 g/L of HemNPs stock suspension was synthesized through the forced hydrolysis of FeCl₃ and used before in previous literature by Huynh et al. From their TEM image of HemNPs, it was found that HemNPs were mostly spherical with some angular feature and had a size of 87 (avg.) nm. For experiments, 8.8 mg/L HemNPs in 1 mM NaCl was prepared from the stock suspension and then sonicated using a bath sonicator (Branson M3800, USA) for 60 minutes to break up aggregates of HemNPs. The carboxylated PSNPs with the nominal size of 104 nm were purchased from Polysciences, Inc. 5 mg/L PSNPs in 1 mM NaCl and 0.2 mM NaHCO₃ (pH 7.1) solution was prepared for experiments and sonicated using the bath sonicator for five minutes to break up aggregates.

Reagents and Solution Chemistry. All experiments were conducted at 25° C., except for the preparation of RBCm suspension. NaCl and NaHCO₃ electrolyte stock solutions were prepared using ACS-grade chemicals (VWR, PA). A stock solution of 10 g/L cationic poly-L-lysine (PLL) hydrobromide (P-1274, Sigma-Aldrich, St. Louis, Mo.) was prepared in HEPES buffer solution made up of 10 mM N-(2-hydroxyethyl) piperazine-N′-(2-ethanesulfonic acid) (HEPES) (H4034, Sigma-Aldrich, St. Louis, Mo.) and 100 mM NaCl. Both HEPES and PLL (molecular weight of 70,000-150,000) stock solutions were filtered through 0.2 μm polypropylene syringe filters (VWR, PA). All experiments with PSNPs were conducted at 1 mM NaCl and pH 7.1 buffered with 0.2 mM NaHCO₃. All experiments with HemNPs were conducted at 1 mM NaCl and pH 5.10±0.02 (adjusted by 10 μM HCl). All solutions were prepared with DI water (MilliporeSigma, MA) with a resistivity of 18.2 MS/cm.

Preparation of Colloidal Suspension of RBCm Fragments from Human Blood. The whole blood (blood group of 0 negative) was purchased from the Continental Blood Bank (Fort Lauderdale, Fla.). The samples were preserved with dipotassium ethylenediaminetetraacetic acid (K₂EDTA) to prevent coagulation of the blood cells. At first, the RBC pellets were isolated from plasma by centrifuging (centrifuge 40R, Thermo Scientific, Danville, Ind.) the whole blood at 4° C. and 800 g for 10 min. The resulting precipitates of erythrocytes were collected and washed three times through centrifugation, withdrawing supernatants, and refilling with phosphate buffered saline (PBS) (Amresco Inc., OH) at pH 7.4. Then, 4-time diluted PBS (0.25×PBS) was added to trigger hemolysis of erythrocytes. The hemolyzed solution was centrifuged at 2500 g and 4° C. for 10 min to separate the cellular contents (i.e., hemoglobin) from the RBC membrane. The supernatant was discarded via a micropipette. The process of refilling with 0.25×PBS, centrifugation, and discarding supernatant was repeated three times. The resulting ghosts of RBCm was further washed with DI water three times using the above process. Vortex was used to redisperse the settled RBC ghosts every time during washing with either 0.25×PB S or DI water.

After the hypotonic treatment using 0.25×PBS, less than 1 mL of pink RBC ghosts were produced and after DI water washing RBC ghosts turned white in color. The centrifuge tubes containing white RBC ghosts in DI water were kept at 3° C. and allowed for static diffusion for 3 days until the erythrocyte's membranes spontaneously dispersed into the DI water and formed the homogenous stock of RBCm suspension. To preserve the RBCm suspension for longer duration, they were stored at −20° C. Approximately, 10 ml RBCm suspension was harvested from 6 ml whole blood resulting in the membrane concentration in RBCm suspension equal to 60% of that in whole blood. Before use, NaCl and NaHCO₃ were added to the 10 mL stock RBCm suspension to have 1 mM NaCl and 0.2 mM NaHCO₃ at pH 7.1. Then, the RBCm suspension was sonicated with probe sonicator (Q55, Qsonica, Newtown, Conn.) for ten cycles of 10 s sonication to further reduce the size of RBCm pieces.

Quartz Crystal Microbalance with Dissipation Monitoring (QCM-D). The formation of SRBCm and the deposition of NPs on SRBCm were conducted using a QCM-D (E1, Q-Sense, Västra Frölunda, Sweden) with QFM 401 flow module. A 5-MHz AT-cut quartz crystal silica-coated (QSX 303, Q-Sense) sensor was mounted in the module.

In order to conduct deposition experiments and derive the deposition attachment efficiencies of NPs on SRBCm, a SRBCm was first developed on the surface of a silica-coated crystal sensor. In order to acquire the deposition kinetics of PSNPs and HemNPs on SRBCm in their corresponding electrolyte solution, both deposition experiments on SRBCm and favorable deposition experiments were performed.

For deposition experiments on SRBCm, the crystal surface was first coated by PLL by successive rinsing of the surface with HEPES buffer, 0.1 g/L PLL solution, and additional HEPES buffer. During the PLL adsorption process, there were sharp decrease and increase of the frequency and dissipation, respectively until they reached a plateau suggesting the crystal surface is completely coated with PLL. Then, the PLL layer was covered by RBCm coating. After rinsing with the corresponding background solution, NP suspension was introduced into measurement chamber and deposition of NPs on SRBCm took place. The favorable deposition experiments of cationic HemNPs were conducted on bare silica surface. The negatively charged silica surface was rinsed by 1 mM NaCl (pH 5.1) to get good baselines. Then, the HemNPs suspension was directed across the silica surface for deposition to occur at 1 mM NaCl and pH 5.1. For PSNPs, the favorable deposition was conducted on PLL-modified silica surface at 1 mM NaCl and 0.2 mM NaHCO₃.

Cryogenic Transmission Electron Microscopy (Cryo-TEM). Cryo-TEM imaging was employed to examine the RBCm fragments in the erythrocyte membrane suspension at 1 mM NaCl and 0.2 mM NaHCO₃. Three microliters of the suspension were applied to carbon grids (Protochips, Inc., Morrisville, N.C.) and vitrified using a Vitrobot (Mark IV, FEI Co., Hillsboro, Oreg.) which operated at 4° C. and ˜90% humidity in the control chamber. Then, the vitrified sample was stored under liquid nitrogen and transferred into a cryo-holder (Model 626/70, Gatan, Inc., Pleasanton, Calif.) for imaging. The sample was inspected using a camera (4k×4k CCD, Gatan, Inc., Pleasanton, Calif.) on a TEM (Tecnai G2 F20-TWIN, FEI Co., Hillsboro, Oreg.) operated at a voltage of 200 kV using low dose conditions (˜20 e/Å2). Images were recorded with a defocus of approximately −3 μm to improve contrast.

Atomic Force Microscopy (AFM) Imaging. AFM imaging of SRBCm and bare silica surface were performed using an atomic force microscope (5420, Agilent Technologies, Inc, Santa Clara, Calif.) in aqueous solutions (i.e., RBCm suspension for SRBCm or DI water for silica surface). A cleaned silica quartz crystal sensor was soaked in 0.1 g/L PLL for 20 min and then subsequently soaked in RBCm suspension for 30 min to form SRBCm on the PLL-modified sensor before observation. Bare silica surface was just soaked in DI water for imaging. The petri dish with the prepared sensor was mounted on the stage of AFM. Triangular silicon nitride cantilever with a nominal spring constant of 0.088 N/m (HYDRA4V-100NG, Applied Nanostructures, Inc., Mountain View, Calif.) was mounted in the AFM cell. The images were acquired in AC mode with 30% drive, a scanning speed of 2.02 in/s, and manual tuning. The images were further processed using Pico image tool.

### Example 2: Preparation and Characterization of RBC Membrane Suspension

In order to develop a supported RBCm coating on the QCM sensor, RBCm suspension was first prepared. Erythrocytes were isolated from plasma and buffy coat after centrifugation of whole blood, and then washed 3 times by phosphate-buffered saline (PBS) followed by hemolysis and hypotonic treatment in 4-time diluted PBS. The remaining RBCm ghosts were sequentially washed by 4-time diluted PBS and deionized (DI) water before they were stored in DI water under static condition at 3° C. in the refrigerator for 3 days. The RBCm ghosts had been fully dispersed into colloidal fragments after the 3-day static diffusion. Right before use, probe sonication was applied to further reduce the size of RBCm fragments and the solution chemistry of the suspension was adjusted to be 1 mM NaCl and 0.2 mM NaHCO₃. The final concentration of RBCm in the working suspensions was equal to 60% of the RBCm concentration in the original whole blood. Dispersed RBC membranes in a solution of 1 mM NaCl and 0.2 mM NaHCO₃ (pH 7.1) after sonication looks like a translucent colloid suspension. The cryogenic transmission electron microscopy (cryo-TEM) image of RBCm suspension shows that erythrocyte membrane had been broken off into colloidal fragments in the suspension. The fragments were amorphous in size or shape. Some fragments were overlapped, developing RBCm aggregates. Their sizes were ca. 450 nm in short diameter and ca. 600 nm in long diameter. The hydrodynamic diameter of the RBCm colloidal fragments in the suspension was determined by dynamic light scattering (DLS) to be 390±90 nm (avg.±SD.). The zeta potential of RBCm fragments was determined to be −0.53±0.41 mV (avg.±SD.) in the solution of 1 mM NaCl and 0.2 mM NaHCO₃, at pH 7.1.

### Example 3: Formation of Supported RBCm on a Silica-Coated Piezoelectric Sensor

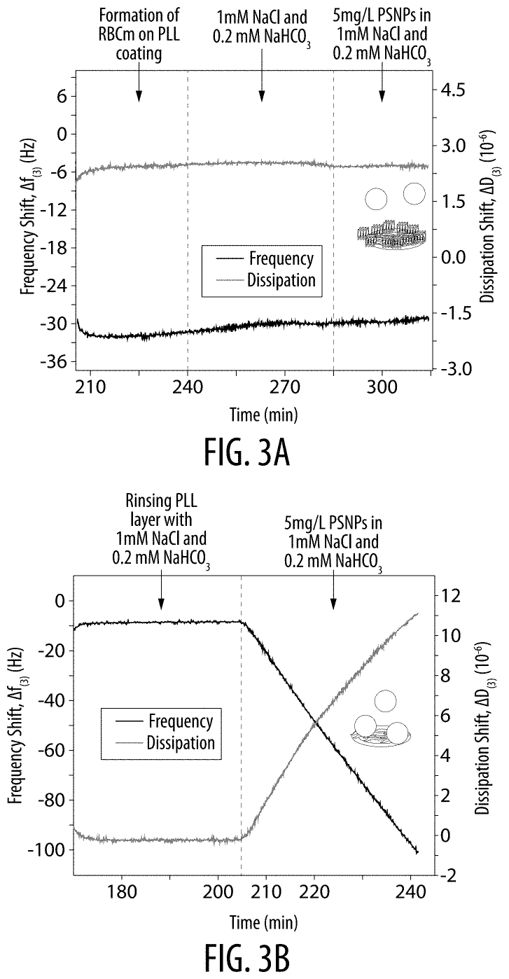

The supported RBCm (SRBCm) layer was developed on a silica-coated piezoelectric sensor of a quartz crystal microbalance with dissipation monitoring (QCM-D) instrument. FIG. 2 presents the representative normalized frequency and dissipation shifts at the third overtone, denoted as Δf₍₃₎ (=Δf₃/3) and ΔD₍₃₎, respectively, of the sensor during the formation of the SRBCm. A stable baseline was first obtained by rinsing the substrate surface with deionized (DI) water at 0.1 mL/min. Then, the negatively charged sensor surface was completely coated by a cationic layer of PLL by sequentially introducing ca. 2 mL of HEPES buffer (i.e., 10 mM N-(2-hydroxyethyl) piperazine-N′-(2-ethanesulfonic acid) (HEPES) and 100 mM NaCl), ca. 2 mL of 0.1 g/L PLL dissolved in HEPES buffer, and ca. 2 mL of HEPES buffer across the sensor surface. After obtaining good baselines of frequency and dissipation by rinsing the PLL-modified surface with a background electrolyte solution of 1 mM NaCl and 0.2 mM NaHCO₃ at pH 7.1, the RBCm suspension at the same solution chemistry was introduced into the measurement chamber.

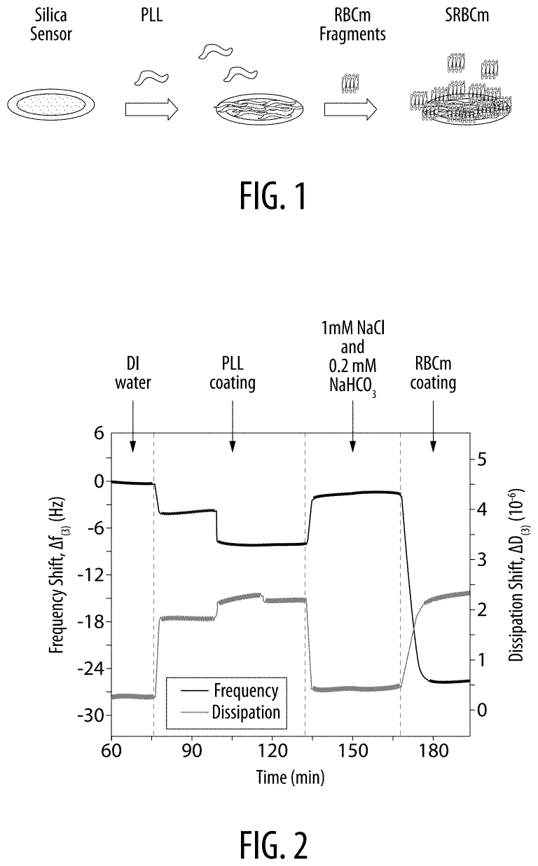

As shown in FIG. 2, Δf₍₃₎ decreased and ΔD₍₃₎ increased sharply due to the deposition of RBCm fragments on PLL-modified sensor surface. As the colloidal fragments of RBC membrane and PLL layer carried negative and positive surface charge, respectively, at pH 7.1, the deposition of RBCm fragments on the PLL-modified surface was favorable due to electrostatic attraction. Both frequency and dissipation attained plateaus in 3 min after starting of the deposition, signifying that, the surface of sensor had been completely coated with RBCm fragments and no multilayer deposition took place. Thus, we speculate that the SRBCm was mainly a single layer of flattened fragments of RBCm which had complete coverage on the surface of PLL modified sensor, as illustrated by the cartoon in FIG. 1. Since RBCm fragments were pieces of original erythrocyte membranes, the SRBCm should retain the important biological components of cell membrane i.e., phospholipid bilayer, sterols, proteins, etc.

The complete coverage of supported RBCm on the PLL-modified silica-coated piezoelectric sensor was verified through deposition experiments of carboxylated polystyrene nanoparticles (PSNPs) on SRBCm.

A supported erythrocyte membrane was developed first. Following the good baseline in the background solution (i.e., 1 mM NaCl and 0.2 mM NaHCO₃), 5 mg/L PSNPs in the same electrolyte solution was introduced across the supported RBCm. No noticeable PSNPs deposition were observed. Thus, both RBCm and PSNPs carried negatively surface charge and the electrostatic repulsion impeded the deposition of PSNPs on the SRBCm coating. On the contrary, in a control experiment the PLL layer was not covered by RBCm.

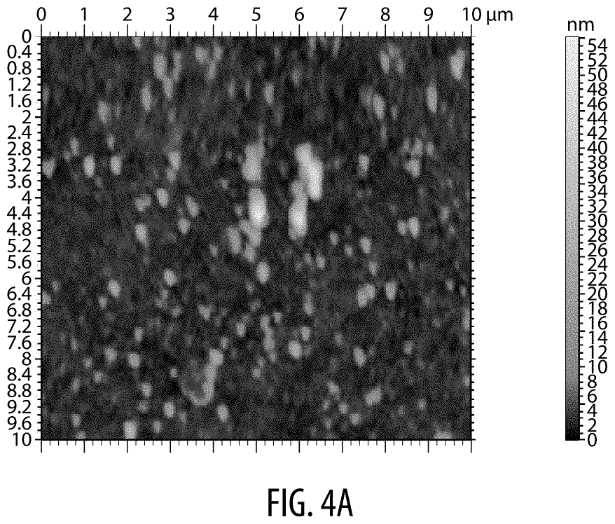

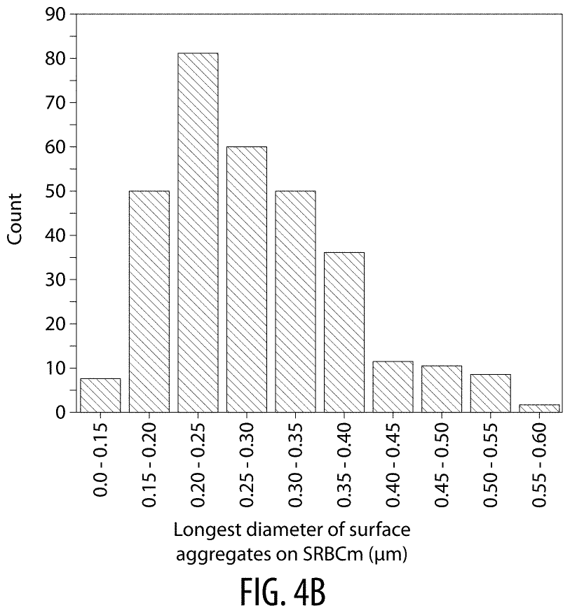

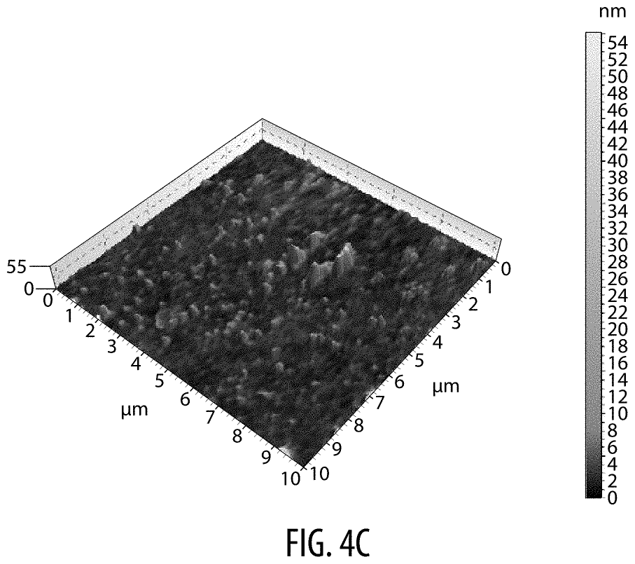

When 5 mg/L PSNPs in 1 mM NaCl and 0.2 mM NaHCO₃ was introduced into the measurement chamber, due to electrostatic attraction PSNPs readily attached to the PLL layer with a favorable deposition rate of 2.48±0.08 (avg.±SD.) Hz/min. The contrast between FIGS. 3A and 3B indicated that there was a complete coverage of RBCm on the PLL-coated surface. Even if there was any pore in the supported RBCm, the diameter of pore should have been less than the size of PSNPs, i.e., 106 nm.

### Example 4: Characterization of Surface Morphology of Supported RBCm using AFM

AFM images of the surface morphology of a supported RBCm on the silica crystal sensor of QCM-D has been obtained in a solution of 1 mM NaCl and 0.2 mM NaHCO₃. Two-dimensional (2D) and three-dimensional (3D) images were taken of the surface morphology of the SRBCm. A control AFM image of bare surface of silica crystal sensor was obtained in DI water. Most of the SRBCm area had the height of a few nanometers which is consistent with the thickness of cell membranes. Thus, consistent with the QCM-D results, the AFM images also show that SRBCm was mainly consisted of a single layer of flattened RBCm. There were also aggregates of RBCm fragments embedded in or deposited on the SRBCm. Some aggregates have the oval shape and some may have a dumbbell-like shape. The range of the longest diameter of the RBCm aggregates was from 120 nm to 590 nm which is consistent with the size of RBCm aggregates observed in cryo-TEM image. The maximum height of aggregates was 53.9 nm. Imaged SRBCm and aggregates of RBCm were stable upon repeated imaging and no lateral movement was detected.

The size distribution of RBCm aggregates shows a mean of the size distribution of 0.28 μm. The distribution is positively skewed (skewness=0.82). In other words, the size data of most membrane fragments are clustered around the left tail of the distribution.

### Example 5: Deposition Kinetics and Attachment Efficiency of Hematite Nanoparticles on Supported RBCm

In order to measure the attachment probability of HemNPs upon collision with the surface of erythrocytes, the deposition attachment efficiency (α_{D}) of HemNPs on SRBCm was derived through QCM-D deposition experiments of HemNPs on both SRBCm and bare silica surface. The α_{D} was calculated using a classic methodology by taking the ratio of shift rate of frequency during the deposition on the SRBCm to that during the favorable deposition on silica. The HemNPs were suspended in the solution of 1 mM NaCl, at pH 5.1.

A SRBCm was first developed on the PLL-modified silica surface as described before. Then, the SRBCm was rinsed with the solutions of 1 mM NaCl and 0.2 mM NaHCO₃ (pH 7.1) and 1 mM NaCl (pH 5.1), sequentially, until the normalized frequency and dissipation responses were stabilized. Afterwards, 8.8 mg/L HemNPs suspended in 1 mM NaCl was introduced into the measurement chamber. Since RBCm in 1 mM NaCl (pH 5.1) had negative zeta potential (−0.78±0.73 mV), deposition occurred when the positively charged HemNPs approached to the negatively charged RBCm surface.

The deposition of the positively charged HemNPs on negatively charged silica surface was conducted as favorable deposition experiments during which the attachment probability of HemNPs was 100% due to the electrostatic attraction between particles and surfaces.

As the deposition of HemNPs proceeded beyond the initial a few minutes, two types of deposition behavior were observed depending on the aggregation propensity of HemNPs, which can be quantified by aggregation attachment efficiency (α_{A}).

Even slight change in the aggregation attachment efficiency could result in significant difference in the deposition behavior of HemNPs on SRBCm. When α_{A}<0.0002, HemNPs had almost no propensity to attach to each other. HemNPs quickly saturated all the available sites on SRBCm. It is speculated that HemNPs quickly saturated and formed a monolayer on the SRBCm at the surface density of 35±15 ng/cm² and no multilayer deposition occurred since HemNPs could not attach to other HemNPs at such low α_{A}. The α_{D} of HemNPs on SRBCm at 1 mM NaCl and pH 5.1, was thus determined to be 0.99±0.85 (avg.±SD.)

When α_{A} was in the range of 0.074 to 0.173, HemNPs had noticeable propensity to, although still low, to attach to other HemNPs. Thus, continuous deposition of HemNPs was observed on SRBCm. The continuous deposition of HemNPs was due to the multilayer deposition of HemNPs on SRBCm.

### Example 6: Deposition Kinetics and Attachment Efficiency of Carboxylated Polystyrene Nanoparticles on Supported RBCm

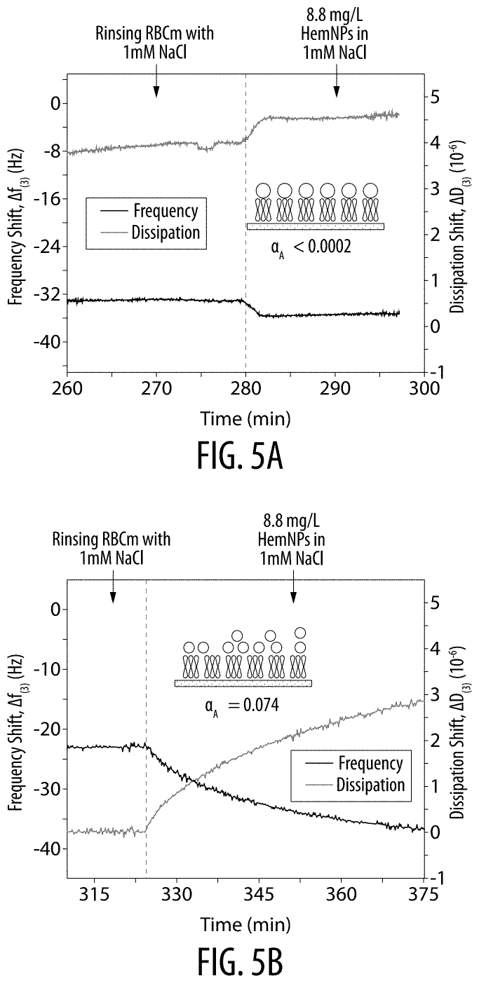

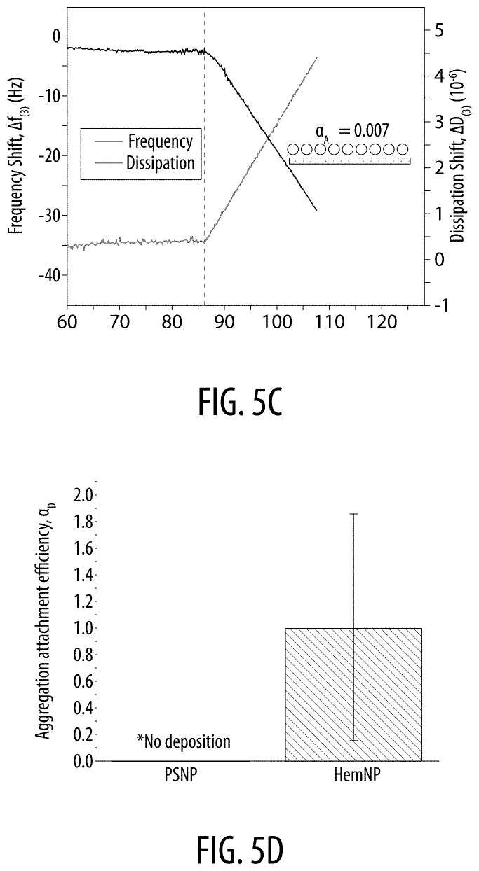

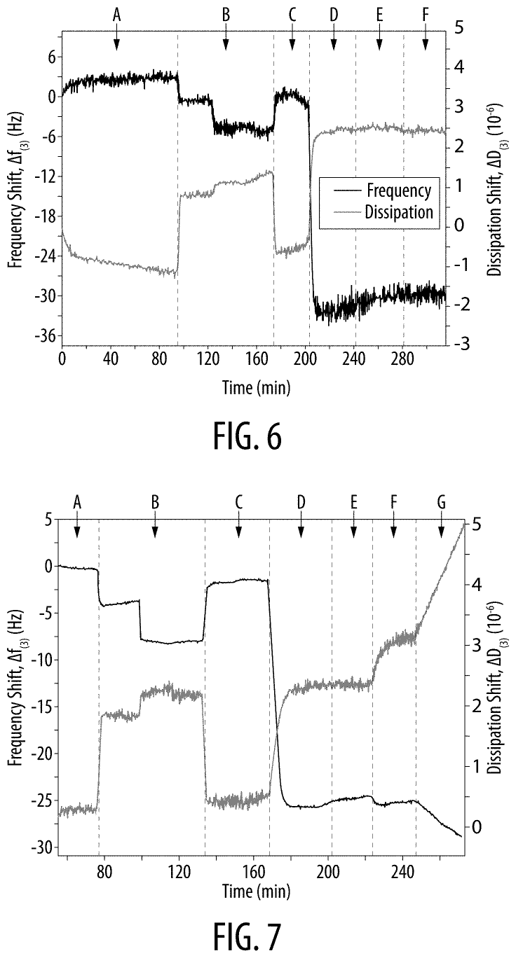

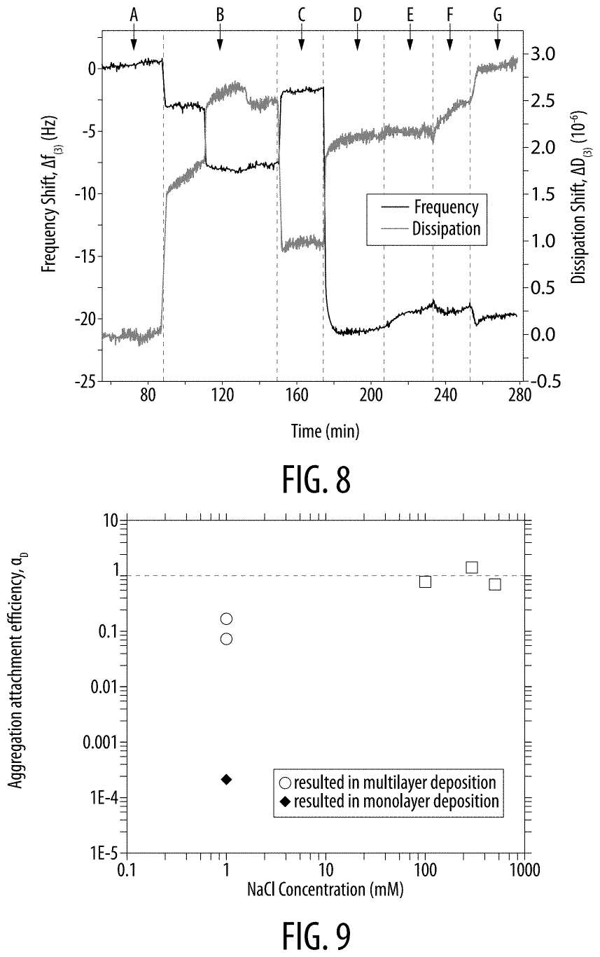

The attachment efficiency of 5 mg/L PSNPs on SRBCm at 1 mM NaCl and 0.2 mM NaHCO₃, pH 7.1 has been derived using the methodology similar to that for HemNPs using the data presented in FIGS. 3A and 3B. As shown by FIG. 3A, no frequency decrease was observed when 5 mg/L PSNPs flowed across SRBCm indicating no deposition of PSNPs upon the interactions with SRBCm. FIG. 6 presents the entire frequency and dissipation data for a duplicate QCM-D experiment which also shows no deposition.

One of the reasons for no deposition of PSNPs on SRBCm was the electrostatic repulsion between them. Moreover, as the head groups of phospholipids are highly hydrophilic, a water layer may have formed on a SRBCm resulting in repulsive hydration force which may also deter the direct contact of PSNPs to SRBCm.

A supported erythrocyte membrane (SRBCm) has been successfully developed on a piezoelectric sensor in the aqueous solution at room temperature. Membranes of RBCs were extracted from whole blood and well dispersed. The dispersed membranes were characterized through cryogenic transmission electron microscopy (cryo-TEM), dynamic light scattering, and zeta potential analysis. The size of the dispersed membrane fragments was 390±90 nm and their zeta potentials were −0.53±0.41 mV at 1 mM NaCl and 0.2 mM NaHCO₃, pH 7.1. The immobilization of membranes was achieved through deposition on the piezoelectric sensor used in a quartz crystal microbalance with dissipation monitoring (QCM-D) system. The frequency shift of −26.2±4.1 Hz and the low ratios of dissipation shift to frequency shift (0.72±0.15×10⁻⁷ Hz⁻¹) suggests the formation of a thin and rigid membrane layer. The SRBCm is comprised of a monolayer of flattened fragments of erythrocyte membranes. The complete coverage of the membrane layer on the sensor was verified through deposition experiments of polystyrene nanoparticles. The surface morphology of the membrane coating was characterized via atomic force microscopy. It was found that aggregates of RBCm with the mean size of 280 nm were present on SRBCm. The deposition attachment efficiencies of model nanoparticles, HemNPs and PSNPs, on SRBCms were obtained in the solution of 1 mM NaCl at pH 5.1 and the solution of 1 mM NaCl and 0.2 mM NaHCO₃, respectively, using a well-established methodology. While PSNPs did not have any deposition, HemNPs had the attachment efficiency of 0.99±0.85 (avg.±SD.). The HemNPs with negligible aggregation propensity (α_{A}<0.0002) quickly saturated the surface of SRBCm at the surface density of 35±15 ng/cm² and no further deposition was observed. In contrast, the HemNPs with noticeable aggregation propensity (α_{A}=0.074 to 0.173) had continuous deposition on SRBCm, probably due to multilayer deposition.

### REFERENCES CITED

[1] Contini, C.; Schneemilch, M.; Gaisford, S.; Quirke, N., Nanoparticle-membrane interactions. Journal of Experimental Nanoscience 2017, 13, (1), 62-81.
[2] Gottschalk, F.; Nowack, B., The release of engineered nanomaterials to the environment. J Environ Monit 2011, 13, (5), 1145-55.
[3] Garner, K. L.; Keller, A. A., Emerging patterns for engineered nanomaterials in the environment: a review of fate and toxicity studies. Journal of Nanoparticle Research 2014, 16, (8).
[4] Malysheva, A.; Lombi, E.; Voelcker, N. H., Bridging the divide between human and environmental nanotoxicology. Nat Nanotechnol 2015, 10, (10), 835-44.
[5] Colvin, V. L., The potential environmental impact of engineered nanomaterials. Nat Biotechnol 2003, 21, (10), 1166-70.
[6] Nel, A.; Xia, T.; Madler, L.; Li, N., Toxic potential of materials at the nanolevel. Science 2006, 311, (5761), 622-7.
[7] Estelrich, J.; Sanchez-Martin, M. J.; Busquets, M. A., Nanoparticles in magnetic resonance imaging: from simple to dual contrast agents. Int J Nanomedicine 2015, 10, 1727-41.
[8] Weissig, V.; Pettinger, T. K.; Murdock, N., Nanopharmaceuticals (part 1): products on the market. Int J Nanomedicine 2014, 9, 4357-73.
[9] Lu, M.; Cohen, M. H.; Rieves, D.; Pazdur, R., FDA report: Ferumoxytol for intravenous iron therapy in adult patients with chronic kidney disease. Am J Hematol 2010, 85, (5), 315-9.
[10] Ventola, C. L., Progress in Nanomedicine: Approved and Investigational Nanodrugs. P T 2017, 42, (12), 742-755.
[11] Kessler, R., Engineered nanoparticles in consumer products: understanding a new ingredient. Environ Health Perspect 2011, 119, (3), a120-5.
[12] Souza, V. G. L.; Fernando, A. L., Nanoparticles in food packaging: Biodegradability and potential migration to food—A review. Food Packaging Shelf 2016, 8, 63-70.
[13] He, X.; Hwang, H. M., Nanotechnology in food science: Functionality, applicability, and safety assessment. J Food Drug Anal 2016, 24, (4), 671-681.
[14] Gupta, R.; Xie, H., Nanoparticles in Daily Life: Applications, Toxicity and Regulations. J Environ Pathol Toxicol Oncol 2018, 37, (3), 209-230.
[15] Kim, K. T.; Eo, M. Y.; Nguyen, T. T. H.; Kim, S. M., General review of titanium toxicity. Int J Implant Dent 2019, 5, (1), 10.
[16] Stearns, R. C.; Paulauskis, J. D.; Godleski, J. J., Endocytosis of ultrafine particles by A549 cells. Am J Respir Cell Mol Biol 2001, 24, (2), 108-15.
[17] Sukhanova, A.; Bozrova, S.; Sokolov, P.; Berestovoy, M.; Karaulov, A.; Nabiev, I., Dependence of Nanoparticle Toxicity on Their Physical and Chemical Properties. Nanoscale Res Lett 2018, 13, (1), 44.
[18] Miller, M. R.; Raftis, J. B.; Langrish, J. P.; McLean, S. G.; Samutrtai, P.; Connell, S. P.; Wilson, S.; Vesey, A. T.; Fokkens, P. H. B.; Boere, A. J. F.; Krystek, P.; Campbell, C. J.; Hadoke, P. W. F.; Donaldson, K.; Cassee, F. R.; Newby, D. E.; Duffin, R.; Mills, N. L., Inhaled Nanoparticles Accumulate at Sites of Vascular Disease. ACS Nano 2017, 11, (5), 4542-4552.
[19] Miller, M. R.; McLean, S. G.; Duffin, R.; Lawal, A. O.; Araujo, J. A.; Shaw, C. A.; Mills, N. L.; Donaldson, K.; Newby, D. E.; Hadoke, P. W., Diesel exhaust particulate increases the size and complexity of lesions in atherosclerotic mice. Part Fibre Toxicol 2013, 10, 61.
[20] Donaldson, K.; Tran, L.; Jimenez, L. A.; Duffin, R.; Newby, D. E.; Mills, N.; MacNee, W.; Stone, V., Combustion—derived nanoparticles: a review of their toxicology following inhalation exposure. Part Fibre Toxicol 2005, 2, 10.
[21] Oberdorster, G. S., Z.; Atudorei, V.; Elder, A.; Gelein, R.;Lunts, A.; Kreyling, W.; Cox, C., Extrapulmonary translocation of ultrafine carbon particles following whole—body inhalation exposure of rats. . J. Toxicol. Environ. Health, Part A 2002, 65, 1531-1543 2002, 65, (20), 1531-43.
[22] Him, S.; Semmler-Behnke, M.; Schleh, C.; Wenk, A.; Lipka, J.; Schaffler, M.; Takenaka, S.; Moller, W.; Schmid, G.; Simon, U.; Kreyling, W. G., Particle size—dependent and surface charge—dependent biodistribution of gold nanoparticles after intravenous administration. Eur J Pharm Biopharm 2011, 77, (3), 407-16.
[23] Li, S. Q.; Zhu, R. R.; Zhu, H.; Xue, M.; Sun, X. Y.; Yao, S. D.; Wang, S. L., Nanotoxicity of TiO(2) nanoparticles to erythrocyte in vitro. Food Chem Toxicol 2008, 46, (12), 3626-31.
[24] Shang, L.; Nienhaus, K.; Nienhaus, G. U., Engineered nanoparticles interacting with cells: size matters. J Nanobiotechnology 2014, 12, 5.
[25] Oberdorster, G.; Oberdorster, E.; Oberdorster, J., Nanotoxicology: an emerging discipline evolving from studies of ultrafine particles. Environ Health Perspect 2005, 113, (7), 823-39.
[26] Villa, C. H.; Anselmo, A. C.; Mitragotri, S.; Muzykantov, V., Red blood cells: Supercarriers for drugs, biologicals, and nanoparticles and inspiration for advanced delivery systems. Adv Drug Deliv Rev 2016, 106, (Pt A), 88-103.
[27] Chen, L. Q.; Fang, L.; Ling, J.; Ding, C. Z.; Kang, B.; Huang, C. Z., Nanotoxicity of silver nanoparticles to red blood cells: size dependent adsorption, uptake, and hemolytic activity. Chem Res Toxicol 2015, 28, (3), 501-9.
[28] de la Harpe, K. M.; Kondiah, P. P. D.; Choonara, Y. E.; Marimuthu, T.; du Toit, L. C.; Pillay, V., The Hemocompatibility of Nanoparticles: A Review of Cell—Nanoparticle Interactions and Hemostasis. Cells 2019, 8, (10).
[29] Cruz, G. G. D. L.; Rodriguez-Fragoso, P.; Reyes-Esparza, J.; Rodriguez-Lopez, A.; Gomez-Cansino, R.; Rodriguez-Fragoso, L., Interaction of Nanoparticles with Blood Components and Associated Pathophysiological Effects. In Unraveling the Safety Profile of Nanoscale Particles and Materials—From Biomedical to Environmental Applications, 2018.
[30] 30. Yi, P.; Chen, K. L., Interaction of multiwalled carbon nanotubes with supported lipid bilayers and vesicles as model biological membranes. Environ Sci Technol 2013, 47, (11), 5711-9.
[31] Zhang, X.; Yang, S., Nonspecific adsorption of charged quantum dots on supported zwitterionic lipid bilayers: real-time monitoring by quartz crystal microbalance with dissipation. Langmuir 2011, 27, (6), 2528-35.
[32] Nel, A. E.; Madler, L.; Velegol, D.; Xia, T.; Hoek, E. M.; Somasundaran, P.; Klaessig, F.; Castranova, V.; Thompson, M., Understanding biophysicochemical interactions at the nano-bio interface. Nat Mater 2009, 8, (7), 543-57.
[33] AshaRani, P. V.; Low Kah Mun, G.; Hande, M. P.; Valiyaveettil, S., Cytotoxicity and genotoxicity of silver nanoparticles in human cells. ACS Nano 2009, 3, (2), 279-90.
[34] Wottrich, R.; Diabate, S.; Krug, H. F., Biological effects of ultrafine model particles in human macrophages and epithelial cells in mono- and co-culture. Int J Hyg Environ Health 2004, 207, (4), 353-61.
[35] Wahab, R.; Khan, F.; Al-Khedhairy, A. A., Hematite iron oxide nanoparticles: apoptosis of myoblast cancer cells and their arithmetical assessment. RSC Advances 2018, 8, (44), 24750-24759.
[36] Hesler, M.; Aengenheister, L.; Ellinger, B.; Drexel, R.; Straskraba, S.; Jost, C.; Wagner, S.; Meier, F.; von Briesen, H.; Buchel, C.; Wick, P.; Buerki-Thurnherr, T.; Kohl, Y., Multi—endpoint toxicological assessment of polystyrene nano—and microparticles in different biological models in vitro. Toxicol In Vitro 2019, 61, 104610.
[37] Abdelkhaliq, A.; van der Zande, M.; Punt, A.; Helsdingen, R.; Boeren, S.; Vervoort, J. J. M.; Rietjens, I.; Bouwmeester, H., Impact of nanoparticle surface functionalization on the protein corona and cellular adhesion, uptake and transport. J Nanobiotechnology 2018, 16, (1), 70.
[38] Pan, D. C.; Myerson, J. W.; Brenner, J. S.; Patel, P. N.; Anselmo, A. C.; Mitragotri, S.; Muzykantov, V., Nanoparticle Properties Modulate Their Attachment and Effect on Carrier Red Blood Cells. Sci Rep 2018, 8, (1), 1615.
[39] Zhao, Z.; Ukidve, A.; Gao, Y.; Kim, J.; Mitragotri, S., Erythrocyte leveraged chemotherapy (ELeCt): Nanoparticle assembly on erythrocyte surface to combat lung metastasis. Sci Adv 2019, 5, (11), eaax9250.
[40] Brenner, J. S.; Pan, D. C.; Myerson, J. W.; Marcos-Contreras, O. A.; Villa, C. H.; Patel, P.; Hekierski, H.; Chatterjee, S.; Tao, J. Q.; Parhiz, H.; Bhamidipati, K.; Uhler, T. G.; Hood, E. D.; Kiseleva, R. Y.; Shuvaev, V. S.; Shuvaeva, T.; Khoshnejad, M.; Johnston, I.; Gregory, J. V.; Lahann, J.; Wang, T.; Cantu, E.; Armstead, W. M.; Mitragotri, S.; Muzykantov, V., Red blood cell—hitchhiking boosts delivery of nanocarriers to chosen organs by orders of magnitude. Nat Commun 2018, 9, (1), 2684.
[41] Yoo, J. W.; Irvine, D. J.; Discher, D. E.; Mitragotri, S., Bio-inspired, bioengineered and biomimetic drug delivery carriers. Nat Rev Drug Discov 2011, 10, (7), 521-35.
[42] Avsievich, T.; Popov, A.; Bykov, A.; Meglinski, I., Mutual interaction of red blood cells influenced by nanoparticles. Sci Rep 2019, 9, (1), 5147.
[43] Anselmo, A. C.; Gupta, V.; Zern, B. J.; Pan, D.; Zakrewsky, M.; Muzykantov, V.; Mitragotri, S., Delivering nanoparticles to lungs while avoiding liver and spleen through adsorption on red blood cells. ACS Nano 2013, 7, (12), 11129-37.
[44] Rothen-Rutishauser, B. M.; Schurch, S.; Haenni, B.; Kapp, N.; Gehr, P., Interaction of fine particles and nanoparticles with red blood cells visualized with advanced microscopic techniques. Environ Sci Technol 2006, 40, (14), 4353-9.
[45] Yousefi, N.; Tufenkji, N., Probing the Interaction between Nanoparticles and Lipid Membranes by Quartz Crystal Microbalance with Dissipation Monitoring. Front Chem 2016, 4, 46.
[46] Liu, X.; Chen, K. L., Aggregation and interactions of chemical mechanical planarization nanoparticles with model biological membranes: role of phosphate adsorption. Environmental Science: Nano 2016, 3, (1), 146-156.
[47] Waldie, S.; Lind, T. K.; Browning, K.; Moulin, M.; Haertlein, M.; Forsyth, V. T.; Luchini, A.; Strohmeier, G. A.; Pichler, H.; Maric, S.; Cardenas, M., Localization of Cholesterol within Supported Lipid Bilayers Made of a Natural Extract of Tailor—Deuterated Phosphatidylcholine. Langmuir 2018, 34, (1), 472-479.
[48] Luchini, A.; Nzulumike, A. N. O.; Lind, T. K.; Nylander, T.; Barker, R.; Arleth, L.; Mortensen, K.; Cardenas, M., Towards biomimics of cell membranes: Structural effect of phosphatidylinositol triphosphate (PIP3) on a lipid bilayer. Colloids Surf B Biointerfaces 2019, 173, 202-209.
[49] Reviakine, I.; Simon, A.; Brisson, A., Effect of Ca2+on the Morphology of Mixed DPPC-DOPS Supported Phospholipid Bilayers. Langmuir 2000, 16, (4), 1473-1477.
[50] Hedegaard, S. F.; Derbas, M. S.; Lind, T. K.; Kasimova, M. R.; Christensen, M. V.; Michaelsen, M. H.; Campbell, R. A.; Jorgensen, L.; Franzyk, H.; Cardenas, M.; Nielsen, H. M., Fluorophore labeling of a cell—penetrating peptide significantly alters the mode and degree of biomembrane interaction. Sci Rep 2018, 8, (1), 6327.
[51] Melby, E. S.; Mensch, A. C.; Lohse, S. E.; Hu, D.; Orr, G.; Murphy, C. J.; Hamers, R. J.; Pedersen, J.A., Formation of supported lipid bilayers containing phase—segregated domains and their interaction with gold nanoparticles. Environmental Science: Nano 2016, 3, (1), 45-55.
[52] Himbert, S.; Alsop, R. J.; Rose, M.; Hertz, L.; Dhaliwal, A.; Moran-Mirabal, J. M.; Verschoor, C. P.; Bowdish, D. M.; Kaestner, L.; Wagner, C.; Rheinstadter, M. C., The Molecular Structure of Human Red Blood Cell Membranes from Highly Oriented, Solid Supported Multi-Lamellar Membranes. Sci Rep 2017, 7, 39661.
[53] Rakow, A. L.; Hochmuth, R. M., Effect of heat treatment on the elasticity of human erythrocyte membrane. Biophys J 1975, 15, (11), 1095-100.
[54] Lepock, J. R., Protein Denaturation During Heat Shock. In Thermobiology, 1997; Vol. 19, pp 223-259.
[55] Gershfeld, N. L.; Murayama, M., Thermal instability of red blood cell membrane bilayers: temperature dependence of hemolysis. J Membr Biol 1988, 101, (1), 67-72.
[56] Yi, P.; Chen, K. L., Influence of surface oxidation on the aggregation and deposition kinetics of multiwalled carbon nanotubes in monovalent and divalent electrolytes. Langmuir 2011, 27, (7), 3588-99.
[57] Cho, N. J.; Frank, C. W.; Kasemo, B.; Hook, F., Quartz crystal microbalance with dissipation monitoring of supported lipid bilayers on various substrates. Nat Protoc 2010, 5, (6), 1096-106.
[58] Richter, R. P.; Berat, R.; Brisson, A. R., Formation of solid—supported lipid bilayers: an integrated view. Langmuir 2006, 22, (8), 3497-505.
[59] Jaspard, F.; Nadi, M.; Rouane, A., Dielectric properties of blood: an investigation of haematocrit dependence. Physiol Meas 2003, 24, (1), 137-47.
[60] Hussain, K. A. SELF-ASSEMBLING OF NEUTRAL AND CHARGED NANOPARTICLES INTO CORESHELL NANOHYBRIDS THROUGH HETEROAGGREGATION WITH SIZE CONTRO. Florida Atlantic University, Boca Raton, Fla., 2019.
[61] Yi, P.; Chen, K. L., Influence of Surface Oxidation on the Aggregation and Deposition Kinetics of Multiwalled Carbon Nanotubes in Monovalent and Divalent Electrolytes. Langmuir 2011, 27, (7), 3588-3599.
[62] Chen, K. L.; Elimelech, M., Aggregation and deposition kinetics of fullerene (C-60) nanoparticles. Langmuir 2006, 22, (26), 10994-11001.
[63] Chen, K. L.; Elimelech, M., Interaction of Fullerene (C-60) Nanoparticles with Humic Acid and Alginate Coated Silica Surfaces: Measurements, Mechanisms, and Environmental Implications. Environ Sci Technol 2008, 42, (20), 7607-7614.
[64] Chen, K. L.; Mylon, S. E.; Elimelech, M., Aggregation kinetics of alginate—coated hematite nanoparticles in monovalent and divalent electrolytes. Environ Sci Technol 2006, 40, (5), 1516-1523.
[65] Chen, K. L.; Elimelech, M., Aggregation and deposition kinetics of fullerene (C60) nanoparticles. Langmuir 2006, 22, (26), 10994-1001.
[66] Sauerbrey, G., Verwendung von Schwingquarzen zur Wgung dnner Schichten and zur Mikrowgung. Zeitschrift fr Physik 1959, 155, (2), 206-222.
[67] Kubiak, K.; Adamczyk, Z.; Ocwieja, M., Kinetics of silver nanoparticle deposition at PAH monolayers: reference QCM results. Langmuir 2015, 31, (10), 2988-96.
[68] Wu, T. H.; Liao, S. C.; Chen, Y. F.; Huang, Y. Y.; Wei, Y. S.; Tu, S. J.; Chen, K. S., Determination of functionalized gold nanoparticles incorporated in hydrophilic and hydrophobic microenvironments by surface modification of quartz crystal microbalance. Appl Surf Sci 2013, 274, 418-424.
[69] Higgins, M. J.; Polcik, M.; Fukuma, T.; Sader, J. E.; Nakayama, Y.; Jarvis, S. P., Structured water layers adjacent to biological membranes. Biophys J 2006, 91, (7), 2532-42.
[70] Berkowitz, M. L.; Vacha, R., Aqueous solutions at the interface with phospholipid bilayers. Acc Chem Res 2012, 45, (1), 74-82.
[71] Berkowitz, M. L.; Bostick, D. L.; Pandit, S., Aqueous solutions next to phospholipid membrane surfaces: insights from simulations. Chem Rev 2006, 106, (4), 1527-39.
[72] Mylon, S. E.; Chen, K. L.; Elimelech, M., Influence of natural organic matter and ionic composition on the kinetics and structure of hematite colloid aggregation: implications to iron depletion in estuaries. Langmuir 2004, 20, (21), 9000-6.
[73] Huynh, K. A.; McCaffery, J. M.; Chen, K. L., Heteroaggregation of multiwalled carbon nanotubes and hematite nanoparticles: rates and mechanisms. Environ Sci Technol 2012, 46, (11), 5912-20.
[74] Ustundag-Okur, N.; Gokce, E. H.; Bozbiyik, D. I.; Egrilmez, S.; Ozer, O.; Ertan, G., Preparation and in vitro—in vivo evaluation of ofloxacin loaded ophthalmic nano structured lipid carriers modified with chitosan oligosaccharide lactate for the treatment of bacterial keratitis. Eur J Pharm Sci 2014, 63, 204-15.
[75] de Kerchove, A. J.; Elimelech, M., Formation of polysaccharide gel layers in the presence of Ca²⁺ and K⁺ ions: measurements and mechanisms. Biomacromolecules 2007, 8, (1), 113-21.
[76] Chen, Q.; Xu, S.; Liu, Q.; Masliyah, J.; Xu, Z., QCM-D study of nanoparticle interactions. Adv Colloid Interface Sci 2016, 233, 94-114.
[77] Yi, P.; Chen, K. L., Influence of solution chemistry on the release of multiwalled carbon nanotubes from silica surfaces. Environ Sci Technol 2013, 47, (21), 12211-8.

What is claimed is:

1. An analytic surface comprising:
a supported red blood cell membrane coating comprising a layer of red blood cell membrane fragments derived from human red blood cells, wherein said red blood cell membrane fragments form a continuous layer on a surface of an analytic substrate.

2. The analytic surface of claim 1, wherein the red blood cell membrane fragments form a continuous layer on the surface of the analytic substrate in an aqueous solution.

3. The analytic surface of claim 1, wherein the red blood cell membrane fragments form a continuous layer on the surface of the analytic substrate without raising the temperature higher than about 37° C.

4. The analytic surface of claim 1, wherein the supported red blood cell membrane coating is formed without a drying process.

5. The analytic surface of claim 1, wherein the supported red blood cell membrane coating mainly comprises flattened fragments of red blood cell membranes derived from human red blood cells and partially comprises aggregates of fragments of red blood cell membrane fragments derived from human red blood cells.

6. The analytic surface of claim 1, wherein the analytic surface is negatively charged.

7. The analytic surface of claim 1, wherein the analytic surface is a piezoelectric sensor.

8. The analytic surface of claim 7, wherein the piezoelectric sensor is first modified by coating with a positively charged substance to form a positively charged surface of the piezoelectric sensor, before deposition of the red blood cell membrane coating.

9. The analytic surface of claim 7, wherein the piezoelectric sensor is first modified by coating with poly-L-lysine (PLL).

10. The analytic surface of claim 7, wherein the supported red blood cell membrane coating is developed on the silica-coated piezoelectric sensor with a negatively charged surface of a quartz crystal microbalance with dissipation monitoring (QCM-D) instrument.

11. The analytic surface of claim 1, wherein the supported red blood cell membrane is used for toxicity screening for nanoparticles, nanodrug delivery, or as a biosensor.

12. A method of providing the analytic surface of claim 1, the method comprising coating the analytic substrate in a suspension of red blood cell membrane fragments.

13. The method of claim 12, wherein the method is performed at a temperature not higher than about 37° C.

14. The method of claim 13, wherein the method does not comprise a drying process.

15. The method of claim 13, wherein the method comprises:
selecting the analytic surface on which to form the supported red blood cell membrane, wherein the analytic surface is negatively charged;
obtaining a stable baseline by rinsing the analytic substrate with deionized water at 0.1 mL/min;
coating a negatively charged analytic surface with a cationic layer of a positively charged substance; and
depositing a red blood cell membrane coating, wherein the red blood cell membrane is comprised of membrane fragments of human red blood cells.

16. The method of claim 13, wherein the negatively charged analytic substrate is a piezoelectric sensor with a negatively charged surface of a quartz crystal microbalance with dissipation monitoring (QCM-D) instrument.

17. The method of claim 13, wherein the cationic layer of a positively charged substance is poly-L-lysine.
